(19) United States
(12) Patent Application Publication
(10) Pub. No.: US 2006/0199992 A1
(43) Pub. Date: Sep. 7, 2006

(54) Magnetic stimulator

(76) Inventors: Solomon R Eisenberg, Newton, MA (US); Daniel Mocanu, Braila (RO)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/549,965

(22) PCT Filed: Mar. 16, 2004
(86) PCT No.: PCT/US04/08007

§ 371 Date: Sep. 16, 2005

Related U.S. Application Data
(60) Provisional application No. 60/455,309, filed on Mar. 17, 2003.

(51) Int. Cl. A61N 1/00

(52) U.S. Cl. 600/14

(57) ABSTRACT

At least two coils deliver at least two time-varying magnetic fields to a target region within a body. The coils are oriented such that the magnetic fields create intersecting electric fields in the target region. The magnetic fields operate at different frequencies and thus produce a beat frequency signal where the electric fields intersect. The frequencies are chosen so a time-varying electric field, or a current induced by a time-varying magnetic field, alternating at the beat frequency would stimulate excitable tissue located in the target region. Some embodiments utilize a novel coil, which includes a first conductor and at least one second conductor electrically connected to the first conductor at a point. The second conductor extends from the point of connection with the first conductor to a location spaced from the first conductor. At least a portion of the second conductor adjacent the point of connection with the first conductor is non-parallel to the first conductor.

### CROSS REFERENCE TO RELATED APPLICATIONS

This application claims the benefit of U.S. Provisional Application No. 60/455,309, filed Mar. 17, 2003, the contents of which are hereby incorporated by reference herein.

### STATEMENT REGARDING FEDERALLY SPONSORED RESEARCH OR DEVELOPMENT

(Not applicable)

### BACKGROUND OF THE INVENTION

1. Field of the Invention

The present invention relates to electrical stimulation of tissues for therapeutic, diagnostic or experimental purposes and, more particularly, to systems that use time-varying magnetic fields to create electric fields or currents that stimulate these tissues.

2. Related Art

Electric and magnetic signals are used to stimulate regions of bodies for therapeutic, diagnostic and experimental purposes. For example, motor-control regions deep within the brains of Parkinson's patients are sometimes electrically stimulated to arrest shaking (dyskinesia), and some protocols for treating depression call for electrically stimulating a certain part of the brain.

Stimulating a brain with pulsed sinusoidal electrical signals can temporarily block or inhibit a brain function. Cognitive neuroscientists have used such stimulation to “knockout” or “temporary lesion” portions of brains to experimentally determine or confirm which parts of the brains control various body parts or functions.

Repeated stimulation of a neuron can produce long-term changes in the neuron. Low-frequency electrical stimulation can cause long-term depression (LTD) of the neuron, which diminishes efficiency of intercellular links. On the other hand, high-frequency stimulation can cause long-term potentiation (LTP) of the neuron. Thus, it may be possible to selectively increase or decrease the excitability of neurons in discrete brain regions and thereby “program” or “reprogram” brain neural circuitry. The possibility of using LTD and LTP to reprogram brain neural circuitry, such as to enable the brain to perform a function that was lost due to a stroke, is presently motivating research in this area.

Electrical stimulation of tissue below a subject's skin is, however, invasive, in that it requires implanting electrodes and sometimes involves risks associated with anesthesia. Fortunately, magnetic pulses are known to induce electric fields and currents that can stimulate excitable tissues, such as nerve cells and muscles. Thus, magnetic pulses can be used to non-invasively stimulate these tissues. A magnetic stimulation field is typically generated by a current-carrying coil. Most successful transcranial magnetic stimulation (TMS) applications involve figure-8 coils. Circular coils have also been used, but the currents they induce in tissues are typically more diffuse.

With conventional coil designs, magnetic field strength drops off sharply with distance from the coil. Increasing the magnetic field strength to overcome this drop-off can have undesirable side effects, including stimulating or over-stimulating surface and near-surface tissue, which can cause skin or muscle twitching or pain. Consequently, magnetic stimulation cannot be effectively used deeper than about 2-3 cm within a body. Unfortunately, many regions of the brain and other potentially beneficial or interesting stimulation regions lie deeper than 2-3 cm and are, therefore, unreachable by conventional magnetic stimulation technology.

Furthermore, conventional magnetic stimulation technology cannot stimulate a region below a body's surface without also stimulating tissue that lies between the surface and the region that is to be stimulated. This lack of ability to target or focus magnetic stimulation can pose problems, such as when it is desirable to stimulate a region deep within a brain without also stimulating other portions of the brain. Thus, the lack of targeting ability, and the related depth limitation discussed above, severely limit the number of situations in which magnetic stimulation can be used successfully.

### BRIEF SUMMARY OF THE INVENTION

Embodiments of the present invention enable a target region of interest to be magnetically stimulated, without necessarily stimulating adjacent regions or regions that lie between the surface and the target region. Some embodiments of the invention utilize at least two time-varying magnetic fields that create intersecting electric fields in the target region. The region where the electric fields intersect is called an “intersection region.” The magnetic fields, and therefore the electric fields, operate at different frequencies and thus produce a beat frequency electric signal in the intersection region. Each of the at least two magnetic fields operates at a frequency/amplitude combination that does not cause significant tissue stimulation. Thus, it is possible to use field strengths high enough to penetrate deeper within a body than is practical with conventional systems. The frequencies are chosen so the difference between the frequencies, i.e., the beat frequency, stimulates tissue located in the intersection region. More precisely, a time-varying electric field, or a current caused by the time-varying electric field, alternates at the beat frequency and stimulates excitable tissue in the intersection region.

Some embodiments of the invention utilize a novel coil configuration to generate a deep-penetrating magnetic field. The coil includes a first conductor and at least one second conductor electrically connected to the first conductor at a point. The at least one second conductor extends from the point of connection with the first conductor to a location spaced from the first conductor. At least a portion of the second conductor adjacent the point of connection with the first conductor is non-parallel to the first conductor. The coil preferably includes a number of second conductors spaced evenly around the first conductor. In one embodiment, the second conductor is a cone-shaped surface.

### BRIEF DESCRIPTION OF THE SEVERAL VIEWS OF THE DRAWINGS

These and other features, advantages, aspects and embodiments of the present invention will become more apparent to those skilled in the art from the following detailed description of an embodiment of the present invention when taken with reference to the accompanying drawings, in which the first digit of each reference numeral identifies the figure in which the corresponding item is first introduced, and in which:

FIG. 1 is a perspective view of a two-coil embodiment of the present invention being used in a clinical or experimental context;

FIG. 2 is a simplified schematic wiring diagram of the embodiment of FIG. 1;

FIG. 3 is a diagram illustrating a position of an intersection region produced by an embodiment, such as the one illustrated in FIG. 1;

FIG. 4 is a diagram illustrating a shift in position of the intersection region of FIG. 3 as a result of altering one magnetic field strength;

FIG. 5 is a diagram illustrating a position of the intersection region of FIGS. 3 and 4 as a result of altering the angle of the coils;

FIG. 6 is a top view of a possible orientation of two coils and an intersection region, relative to a subject, according to one embodiment of the present invention;

FIG. 7 is a top view of a possible orientation of four-coils and an intersection region, relative to a subject, according to another embodiment of the present invention;

FIG. 8 is a simplified schematic wiring diagram of the embodiment of FIG. 7;

FIG. 9 is an alternative simplified schematic wiring diagram of the embodiment of FIG. 7;

FIG. 10 is a perspective view of a four-coil embodiment of the present invention being used in a clinical or experimental context;

FIG. 11 is a simplified schematic wiring diagram the embodiment of FIG. 10;

FIG. 12 is a diagram of a coil that can be used with the embodiments of FIGS. 1 and 10 or with conventional magnetic stimulation equipment;

FIG. 13 is a diagram of an alternative embodiment of the coil of FIG. 12; and

FIGS. 14A, 14B, 14C, 14D and 14E contain diagrams of other alternative embodiments of the coil of FIG. 12.

### DETAILED DESCRIPTION OF THE INVENTION

Embodiments of the present invention use at least two coils to deliver at least two time-varying magnetic fields to a body. Each magnetic field induces an electric field and electric currents in electrically conductive tissues, such as nerves or muscles, within a portion of the body. Each electric field and its currents may extend beyond its respective magnetic field, because of the conductive nature of the tissues.

The at least two magnetic fields need not necessarily intersect, however the coils are oriented such that the electric fields or currents intersect in a target region of the body. The coils are preferably driven at frequencies and amplitudes that do not directly cause significant tissue stimulation, but a beat frequency signal produced in a region where the electric fields or currents intersect (the intersection region) alternates at a frequency (the beat frequency) that stimulates excitable tissue in the target region.

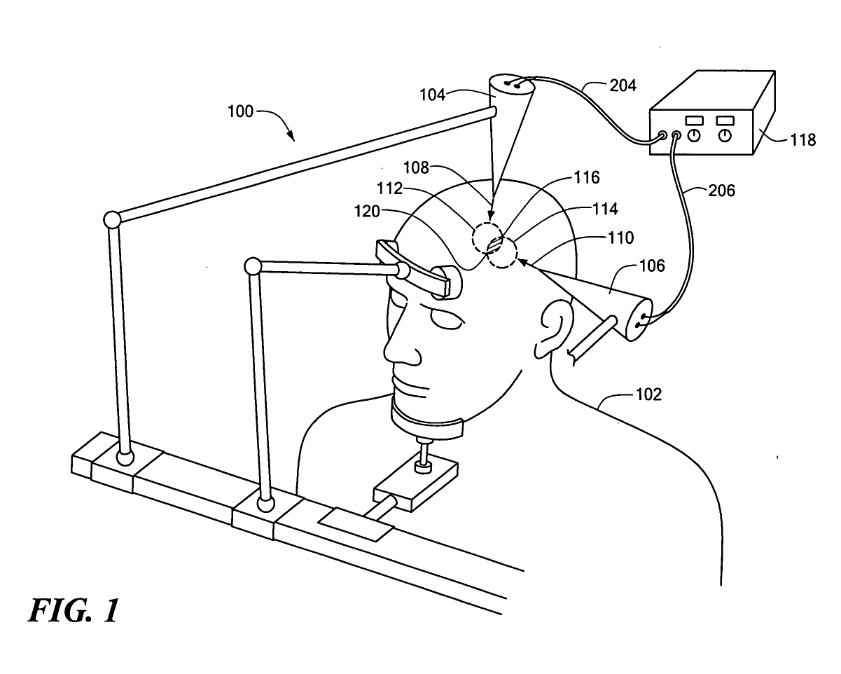

In clinical or experimental contexts, it is often desirable to precisely orient the coils relative to a body part and hold the body part steady, so the electric fields intersect in the target region. Sometimes it is necessary to maintain or establish a coil(s)-to-body part orientation for a period of time during a treatment or repeatedly over a series of treatments. Fixtures, such as the one shown at 100 in FIG. 1, can be used to establish and maintain such a coil(s)-to-body part orientation. Although the fixture 100 is shown being used to hold a head of a subject 102 steady in conjunction with stimulating a region within the subject's head, other similar fixtures (not shown) can be used to hold other body parts steady in conjunction with stimulating other regions within a subject's body. Alternatively, head-fitting coils (so-called “cap” coils) or coils fitted to other body parts can be used. In other embodiments, one or both of the coils can be hand-held.

The coils 104 and 106 produce magnetic fields (indicated by arrows 108 and 110), which induce respective electric fields 112 and 114. As noted, the electric fields 112 and 114 can extend beyond the respective magnetic fields 108 and 110 due to the conductive nature of the tissues. The coils 104 and 106 are oriented so the electric fields 112 and 114 intersect in an intersection region 116. The orientation of the coils 104 and 106 and the strengths of the magnetic fields 108 and 110 are selected to position the intersection region 116 so it corresponds to the target region of the subject 102, as described in more detail below. Embodiments of the invention preferably use a novel coil design, which is described in detail below. Alternatively, conventional figure-8, circular, Helmholtz, Hesed, cap or other types of coils, coil arrays or coil combinations can be used.

The intersection region 116 shown in this example is located within the brain of the subject 102, but the intersection region can be located elsewhere in the subject's head or in another portion of the subject's body. In the example shown in FIG. 1, the magnetic fields 108 and 110 penetrate at least part way through the subject's head. In some applications, the magnetic fields penetrate the brain. A magnetic field is referred to herein as being adjacent a brain whether the magnetic field penetrates the brain or is merely near the brain.

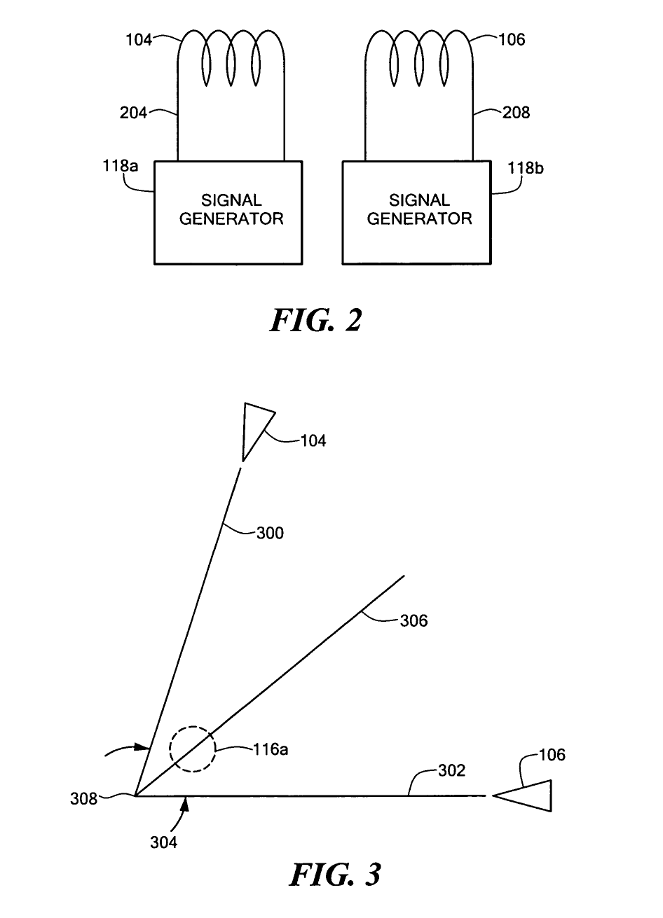

Each coil 104 and 106 is driven by a signal generator 118 to produce its respective time-varying magnetic field 108 and 110. FIG. 2 is a simplified schematic diagram of one embodiment of the present invention. Coil 104 is connected to a first signal generator 118a, preferably by a first flexible cable 204, and coil 106 is connected to a second signal generator 118b, preferably by a second flexible cable 206. The signal generators 118a and 118b include appropriate power supplies, amplifiers, signal strength controls, frequency controls, timers, coil cooling systems, etc. (not shown), as are well-known in the art. Amplitudes of the magnetic fields 108 and 110 vary according to the signals that drive the respective coils 104 and 106. Preferably, the coils 104 and 106 are driven by sinusoidal signals, but other waveforms, such as square waves, are acceptable. The magnetic fields 108 and 110, and therefore the stimulation, can be applied in pulses or continuously for a period of time. The magnetic fields 108 and 110 are preferably pulsed, such as alternatingly on for 10 mSec. and off for 90 mSec., to allow the coils to cool after each pulse.

Returning to FIG. 1, each coil 104 and 106 produces a time-varying magnetic field 108 and 110 that alternates at a different frequency. The frequencies are preferably between about 5 KHz and about 100 KHz, although other frequencies below about 5 KHz or above about 100 KHz are also acceptable. The frequencies and amplitudes are preferably chosen so the magnetic fields 108 and 110, or electric fields or currents they induce, do not significantly directly stimulate tissues within the magnetic fields.

The frequencies are also chosen so a time-varying electric field (or electric currents created by the electric field) alternating at a frequency equal to the difference between the two magnetic field frequencies would stimulate excitable tissue located within the intersection region 116. The difference between the two magnetic field frequencies preferably is between about 10 Hz and about 50 Hz, although differences between about 1 Hz and about 100 Hz or any frequency that would stimulate excitable tissue are acceptable.

As noted, each magnetic field 108 and 110 induces a time-varying electric field 112 and 114. These electric fields 112 and 114 interact in the intersection region 116 to produce the beat frequency time-varying electric field 120. The time-varying electric field 120 alternates at a frequency equal to the difference between the magnetic field frequencies, i.e. the beat frequency.

The location of the intersection region 116 is largely determined by the orientation of the coils 104 and 106 and the strengths of the magnetic fields 108 and 110. As shown in FIG. 3, if the coils 104 and 106 are oriented such that their respective axes 300 and 302 form an angle 304, the intersection region 116a lies along a line 306 that divides the angle. The intersection region 116a is displaced along the line 306, away from the vertex 308 of the angle 304, toward the coils 104 and 106. This displacement and the exact location of the line 306 are influenced by tissues, particularly conductive tissues, that lie within the magnetic fields and electric fields, as well as the coils' designs.

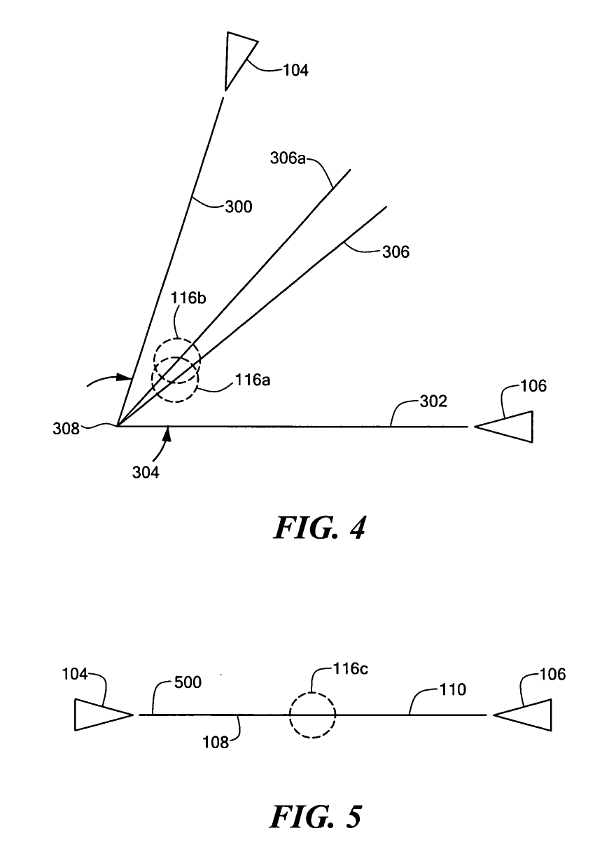

If the magnetic fields 108 and 110 are of equal strengths, the line 306 approximately bisects the angle 304 formed by the coil axes 300 and 302. However, as shown in FIG. 4, if one of the magnetic fields (for example, the field produced by coil 104) is weaker than the other magnetic field, the line 306a and the intersection region 116b are displaced toward the axis of the weaker magnetic field and further away from the vertex 308.

In general, as the angle between the coil axes increases, the intersection region moves closer to the vertex 308. To illustrate this point, FIG. 5 illustrates coils 104 and 106 oriented in opposition, i.e. their respective magnetic fields 108 and 110 are aimed at each other along a common axis 500. In other words, the coils 104 and 106 are oriented 180° apart. If the coils 104 and 106 are oriented in opposition, and the magnetic fields are of equal strengths, the intersection region 116c lies approximately half way between the coils and along the axis 500.

Returning to FIG. 1, the arrows representing the magnetic fields 108 and 110 indicate directions of the respective magnetic fields. The magnetic fields 108 and 110 are oriented generally toward the target region. The coils 104 and 106 are oriented, and the strengths of the magnetic fields 108 and 110 are adjusted, such that the intersection region 116 is preferably approximately the same size as the region of the body that is to be stimulated. However, the intersection region can be larger or smaller than the region to be stimulated.

In general, the strength of the beat frequency electric field 120 is approximately twice the strength of an electric field that would be produced by the weaker of the two magnetic fields 108 or 110 alone. Similarly, electric currents created by the beat frequency electric field 120 are approximately twice the strength of currents that would be produced by the electric field produced by the weaker magnetic field alone. Thus, conventional calculations can be used to determine the strengths of the magnetic fields 108 and 110 needed to stimulate a target region, given the depth of the target region within a body and the desired strength of a stimulating electric field to be applied to the target region.

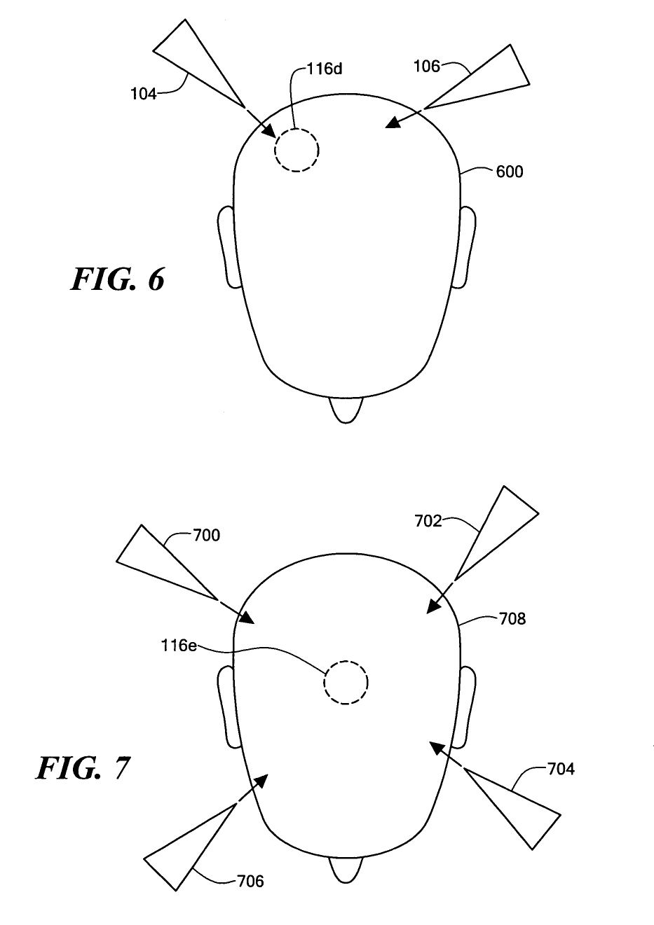

As noted, the coils are oriented about the subject such that the electric fields intersect in the target region. Preferably, the coils are oriented such that the beat frequency electric field 120 does not extend outside the target region or the amount of this out-of-target region extension is minimal. Thus, tissues outside the target region are not stimulated, or out-of-target region stimulation is minimal. FIG. 6 is a top view of two coils 104 and 106 oriented about a head 600 of a subject. The coils 104 and 106 produce magnetic fields that ultimately create a beat frequency electric field or currents in an intersection region 116d.

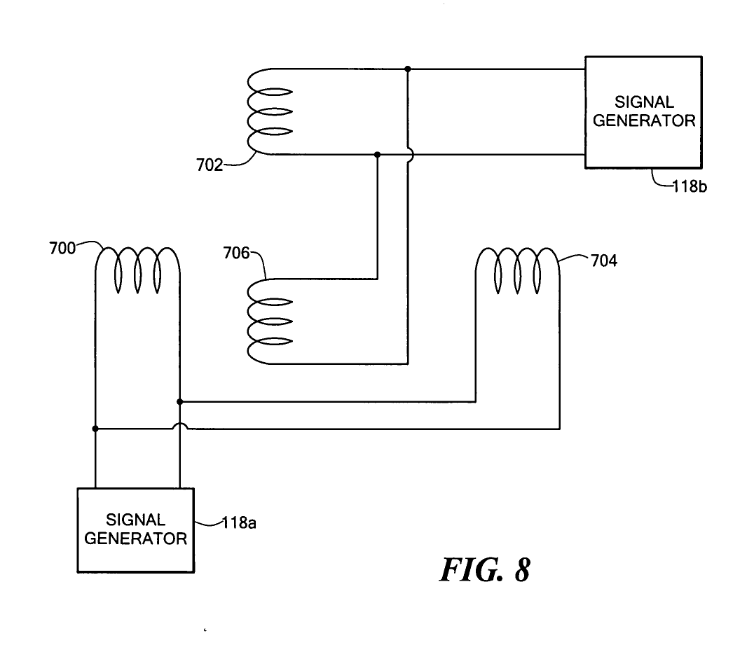

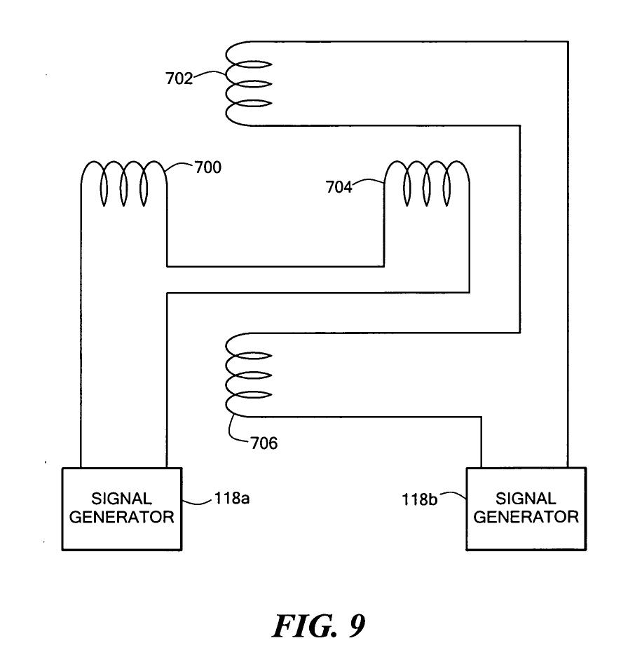

In some embodiments, more than two coils are used to produce the intersecting electric fields. For example, FIG. 7 shows four coils 700, 702, 704 and 706 oriented about a head 708 of a subject to stimulate a target region 116e. If more than two coils are used, as in this example, each of two signal generators can drive one or more of the coils. For example, as shown in FIG. 8, the coils 700 and 704, which are driven by one signal generator 118a, can be connected to each other in parallel, and the coils 702 and 706, which are driven by the other signal generator 118b, can be connected to each other in parallel. Alternatively, as shown in FIG. 9, the coils 700 and 704 can be connected to each other in series, and the other coils 702 and 706 can be connected to each other in series.

As noted, the coils can be Helmholtz or other types of coils. For example, the coils 700 and 704 shown in FIG. 9 can be part of a Helmholtz coil pair, and the other coils 702 and 706 can be part of another Helmholtz coil pair.

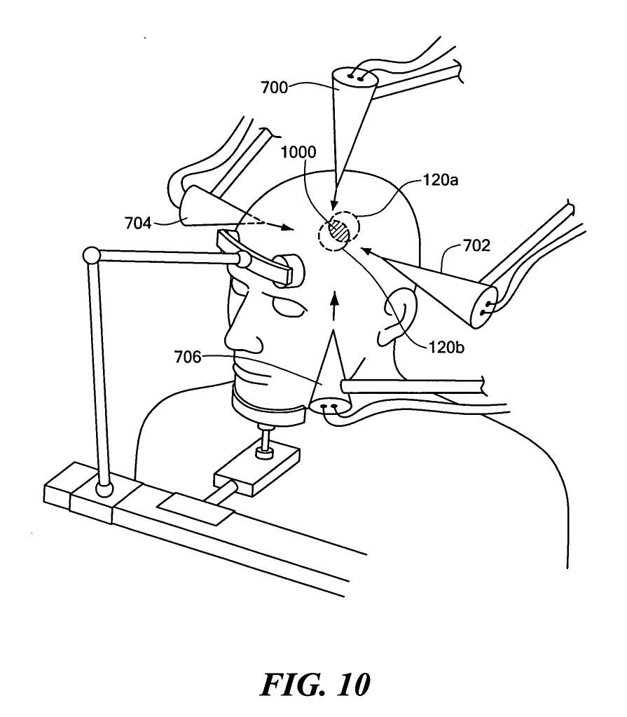

The coils 700, 702, 704 and 706 can be oriented such that all the electric fields produced by the coils intersect. Alternatively, the coils can be oriented such that pairs of electric fields intersect in intersection regions, and the intersection regions fully or only partially overlap each other, as described in more detail below, with reference to FIG. 10.

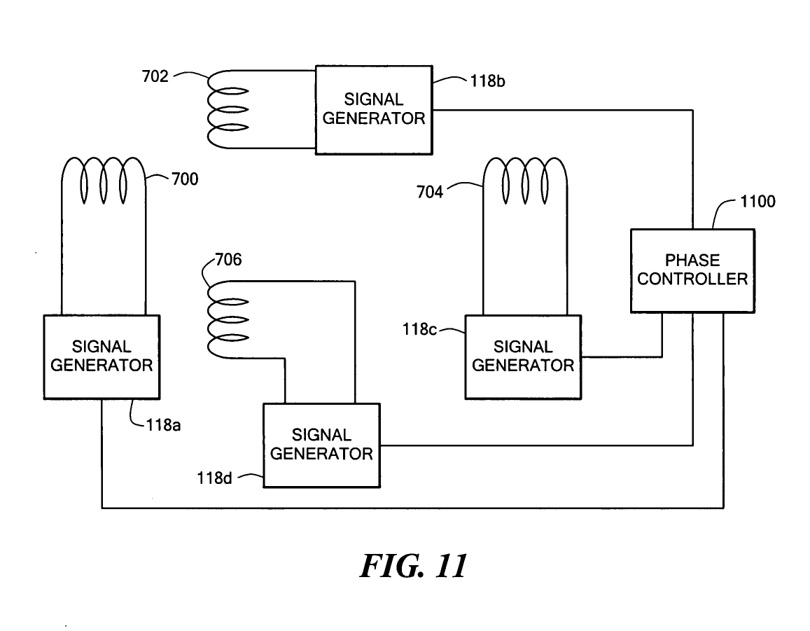

Thus far, embodiments that generate magnetic fields operating at two different frequencies have been discussed. Alternatively, magnetic fields operating at more than two different frequencies can be used. Such an arrangement can, for example, be used when it is difficult or inconvenient to generate a sufficiently strong or sufficiently targeted beat frequency signal using only two frequencies. For example, as shown in FIGS. 10 and 11, each of four coils 700, 702, 704 and 706 can be connected to a respective signal generator 118a, 118b, 118c and 118d.

Two of the signal generators 118a and 118b and two of the coils 700 and 702 can operate at a first pair of frequencies (F1 and F2) to produce a first pair of electric fields that intersect, as described above, to produce a first beat frequency signal 120a. The first beat frequency is the difference between the first pair of frequencies, i.e. the absolute value of (F1-F2).

The other two signal generators 118c and 118d and the other two coils 704 and 706 can operate at a second pair of frequencies (F3 and F4), different than the first pair of frequencies (F1 and F2), to produce a second pair of electric fields that intersect to produce a second beat frequency signal 120b. The second beat frequency is the difference between the second pair of frequencies, i.e. the absolute value of (F3-F4).

The coils can be oriented such that the two beat frequency electric fields 120a and 120b fully or only partially overlap each other. If the beat frequency electric fields 120a and 120b only partially overlap, the maximum stimulation is provided in a region 1000 where the two beat frequency electric fields overlap, and less or no stimulation is provided in the remainder of the two beat frequency electric fields.

The considerations described above, with respect to a two-frequency system, apply to a system that uses more than two frequencies. Each frequency/amplitude combination is preferably chosen so it does not significantly stimulate tissue within the respective field, and the frequencies are chosen so beat frequency signals produced by the electric fields (or currents) stimulate excitable tissue in one or more beat frequency electric fields.

The beat frequencies can be identical or they can be different from each other. If the beat frequencies are identical, it is preferable for the beat frequency signals to be in phase with each other, so they do not destructively interfere with each other. A phase controller 1100 (FIG. 11) can be used to maintain a phase relationship among at least some of the signals generated by the signal generators 118a-d, so the resulting beat frequency signals are in phase.

As discussed above, with conventional coil design, magnetic field strength drops off sharply with distance from the coil. Embodiments of the present invention preferably use a novel coil design that provides deeper magnetic field penetration than conventional coil designs. In addition, this coil can be advantageously used with conventional magnetic stimulation equipment. When the coil is used with conventional magnetic stimulation equipment, it is preferably operated at a frequency between about 10 and 100 Hz, although frequencies between about 1 Hz and 1 KHz, or any frequency that would stimulate excitable tissue, are acceptable.

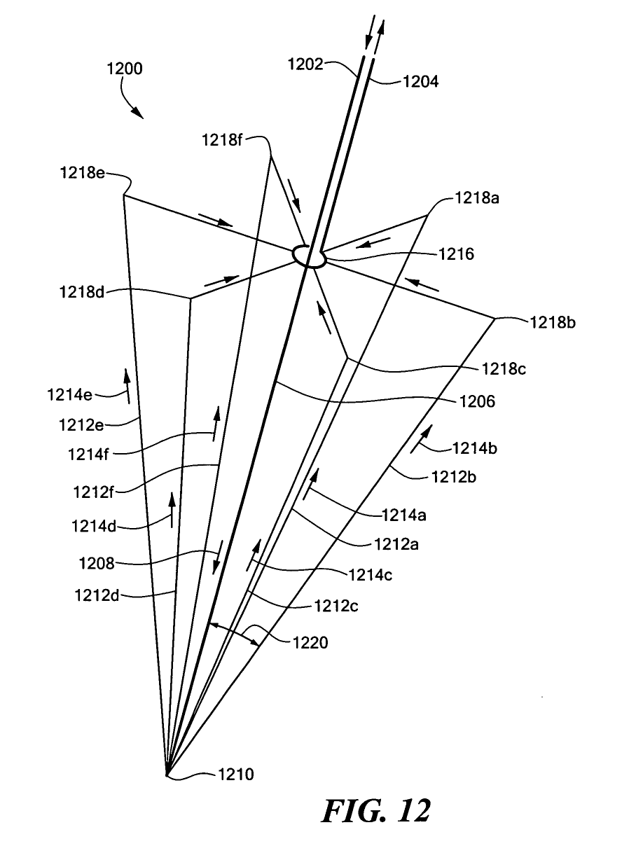

FIG. 12 illustrates one embodiment 1200 of such a coil. The coil 1200 includes two leads 1202 and 1204, by which it can be connected to a signal generator (not shown), such as via a flexible cable (not shown). One lead 1202 is connected to a first conductor 1206, which provides a signal path (indicated by arrow 1208) to a point 1210, preferably at the end of the first conductor. The first conductor 1206 is preferably substantially straight, although a slightly curved first conductor or minor deviations (such as a series of “s” shaped segments) are acceptable.

At least one second conductor (examples of which are shown at 1212a-f) provides a signal path (examples of which are indicated by arrows 1214a-f) from the point 1210. The second conductor 1212 is oriented generally back along the signal path 1208 of the first conductor 1206. The second conductor 1212 is connected to the second lead 1204, such as by a bus 1216. Thus, the second conductor 1212 is connected in series with the first conductor 1206. Together, the first and second conductors 1206 and 1212 provide a continuous signal path through the coil 1200. The first and second conductors 1206 and 1212 can be wires or they can be made from a single piece of wire bent proximate the point 1210.

The second conductor 1212 extends from the point 1210 of connection with the first conductor 1206 to a location (examples of which are shown at 1218a-f) spaced from the first conductor. At least a portion of the second conductor 1212 adjacent the point 1210 of connection (such as the portion between the point 1210 and the location 1218) is non-parallel to the first conductor 1206. From the location 1218, the second conductor extends to the bus 1216, although this extension need not be straight. The second conductor 1212 forms an angle (an example of which is shown at 1220) with the first conductor 1206. This angle 1220 is preferably between about 10° and about 20°, although other angles as small as about 10 are acceptable. Angles up to 45°, 90° or more are also acceptable.

The coil 1200 preferably includes six second conductors 1212 spaced evenly around the first conductor 1206, although fewer (as few as one) or more second conductors 1212 are acceptable. When more than one second conductor is used, electric current flowing along the first conductor 1206 is approximately evenly divided among the second conductors 1212a-f. Thus, the magnetic field surrounding each second conductor 1212 is weaker than the magnetic field surrounding the first conductor 1206.

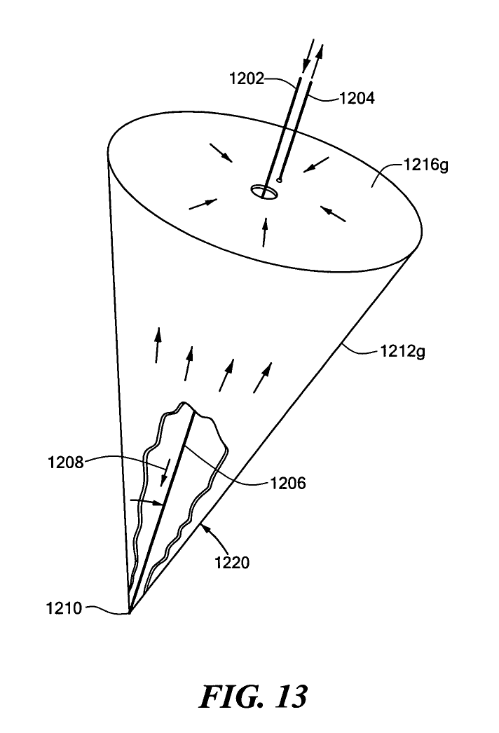

Alternatively, as shown in FIG. 13, the second conductor 1212g can be a surface or a portion of a surface (such as a cone). Bus 1216g can also be a surface or portion thereof.

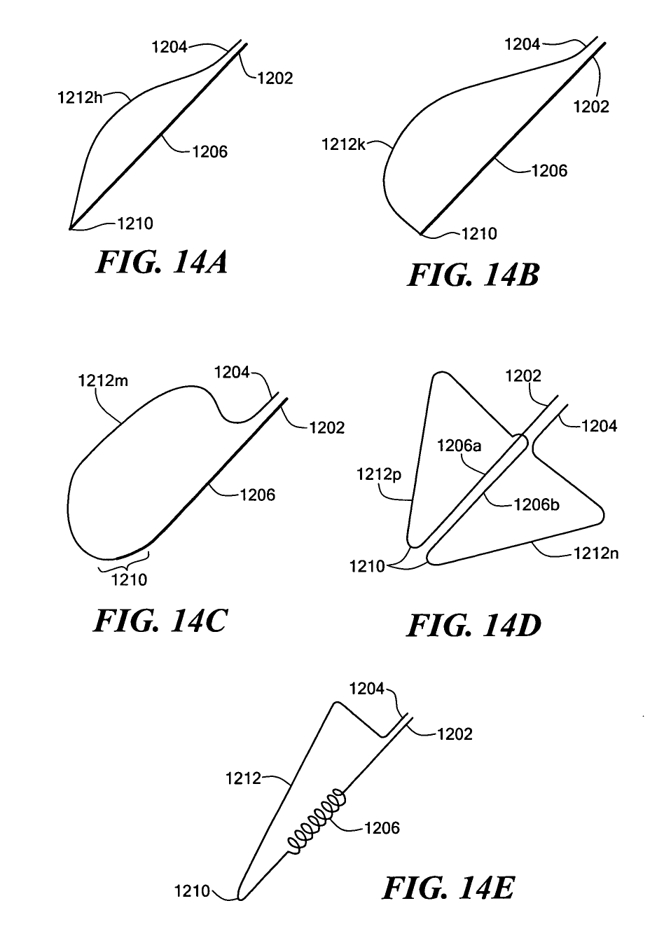

Although FIGS. 12 and 13 show second conductors 1212 that extend substantially straight from the point 1210 of connection with the first conductor 1206 to the location 1218 spaced from the first conductor, other shapes (such as an umbrella shape) are also acceptable. Examples of other acceptable shapes of second conductors are shown in FIGS. 14A-D at 1212h, 1212k, 1212m, 1212n and 1212p. As shown in FIG. 14C, there need not be a definite point at which the first conductor 1206 connects to the second conductor 1212m.

Although FIGS. 12 and 13 show a substantially straight first conductor 1206, other shapes (such as a helical coil, as shown in FIG. 14E) are acceptable. Furthermore, as shown in FIG. 14D, the first conductor can include more than one substantially parallel conductor (examples of which are shown at 1206a and 1206b), and the second conductors (such as 1212n and 1212p) can be connected in series with the first conductors. In addition, features shown in FIGS. 12, 13 and 14A-E can be combined in an embodiment. For example, the six-first-conductor embodiment of FIG. 12 can be constructed with a coiled second conductor.

While the invention has been described with reference to a preferred embodiment, those skilled in the art will understand and appreciate that variations can be made while still remaining within the spirit and scope of the present invention, as described in the appended claims. For example, various types of coils (circular, figure-8, Helmholtz, etc.) can be combined in a single embodiment. In addition, various types or combinations of coils can be combined with two or more signal generators.

What is claimed is:

1. A magnetic stimulator for magnetically stimulating a region of a body, comprising:
a first coil producing a first time-varying magnetic field adjacent a brain of the body at a first frequency; and
a second coil producing a second time-varying magnetic field adjacent the brain at a second frequency that is different than the first frequency;
wherein the first and second coils are oriented such that the first and second magnetic fields produce a beat frequency time-varying electric field in the region of the body, the beat frequency being determined by the first and second frequencies.

2. The magnetic stimulator of claim 1, wherein the first frequency is within about 100 Hz of the second frequency.

3. The magnetic stimulator of claim 1, wherein the first frequency is within about 50 Hz of the second frequency.

4. The magnetic stimulator of claim 1, wherein the first and second frequencies are each between about 5 KHz and about 100 KHz.

5. The magnetic stimulator of claim 1, wherein the beat frequency is between about 1 Hz and about 100 Hz.

6. The magnetic stimulator of claim 1, wherein the beat frequency is between about 10 Hz and about 50 Hz.

7. The magnetic stimulator of claim 1, wherein the first coil comprises a Hesed coil, and the second coil comprises a Hesed coil.

8. The magnetic stimulator of claim 1, wherein the first coil comprises at least two coils, and the second coil comprises at least two coils.

9. The magnetic stimulator of claim 8, wherein the first coil comprises a Helmholtz coil, and the second coil comprises a Helmholtz coil.

10. The magnetic stimulator of claim 8, wherein the at least two coils of the first coil are electrically connected to each other in series, and the at least two coils of the second coil are electrically connected to each other in series.

11. The magnetic stimulator of claim 8, wherein the at least two coils of the first coil are electrically connected to each other in parallel, and the at least two coils of the second coil are electrically connected to each other in parallel.

12. The magnetic stimulation of claim 1, wherein amplitudes of the first and second time-varying magnetic fields vary as respective sine functions.

13. The magnetic stimulator of claim 1, further comprising:
a first signal generator connected to the first coil; and
a second signal generator connected to the second coil.

14. The magnetic stimulator of claim 13, wherein:
the first signal generator produces a signal at the first frequency; and
the second signal generator produces a signal at the second frequency.

15. The magnetic stimulator of claim 1, wherein the first coil comprises:
a first conductor defining a signal path to a point on the first conductor; and
at least one second conductor defining a signal path away from the point, the at least one second conductor being electrically connected in series with the first conductor at the point and extending from the point to a location spaced from the first conductor by a predetermined distance, at least a portion of the at least one second conductor adjacent the point being non-parallel to the first conductor.

16. A magnetic stimulator for magnetically stimulating a region of a body, comprising:
a first coil producing a first time-varying magnetic field at a first frequency; and
a second coil producing a second time-varying magnetic field at a second frequency that is different than the first frequency, the first frequency being within about 50 Hz of the second frequency;
wherein the first and second coils are oriented such that the first and second magnetic fields produce a beat frequency time-varying electric field in the region of the body, the beat frequency being determined by the first and second frequencies.

17. The magnetic stimulator of claim 16, wherein the first and second frequencies are each between about 5 KHz and about 100 KHz.

18. The magnetic stimulator of claim 16, wherein the beat frequency is between about 10 Hz and about 50 Hz.

19. The magnetic stimulator of claim 16, wherein the first coil comprises at least two coils, and the second coil comprises at least two coils.

20. The magnetic stimulator of claim 19, wherein the first coil comprises a Helmholtz coil, and the second coil comprises a Helmholtz coil.

21. The magnetic stimulator of claim 19, wherein the at least two coils of the first coil are electrically connected to each other in series, and the at least two coils of the second coil are electrically connected to each other in series.

22. The magnetic stimulator of claim 19, wherein the at least two coils of the first coil are electrically connected to each other in parallel, and the at least two coils of the second coil are electrically connected to each other in parallel.

23. The magnetic stimulation of claim 16, wherein amplitudes of the first and second time-varying magnetic fields vary as respective sine functions.

24. The magnetic stimulator of claim 16, further comprising:
a first signal generator connected to the first coil; and
a second signal generator connected to the second coil.

25. The magnetic stimulator of claim 24, wherein:
the first signal generator produces a signal at the first frequency; and
the second signal generator produces a signal at the second frequency.

26. The magnetic stimulator of claim 16, wherein the first coil comprises:
a first conductor defining a signal path to a point on the first conductor; and
at least one second conductor defining a signal path away from the point, the at least one second conductor being electrically connected in series with the first conductor at the point and extending from the point to a location spaced from the first conductor by a predetermined distance, at least a portion of the at least one second conductor adjacent the point being non-parallel to the first conductor.

27. A magnetic stimulator for magnetically stimulating a region of a body, comprising:
a first coil producing a first time-varying magnetic field at a first frequency; and
a second coil producing a second time-varying magnetic field at a second frequency that is different than the first frequency, each of the first and second frequencies being between about 5 KHz and about 100 KHz;
wherein the first and second coils are oriented such that the first and second magnetic fields produce a beat frequency time-varying electric field in the region of the body, the beat frequency being determined by the first and second frequencies.

28. The magnetic stimulator of claim 27, wherein the first frequency is within about 100 Hz of the second frequency.

29. The magnetic stimulator of claim 27, wherein the beat frequency is between about 1 Hz and about 100 Hz.

30. The magnetic stimulator of claim 27, wherein the beat frequency is between about 10 Hz and about 50 Hz.

31. The magnetic stimulator of claim 27, wherein the first coil comprises at least two coils, and the second coil comprises at least two coils.

32. The magnetic stimulator of claim 31, wherein the first coil comprises a Helmholtz coil, and the second coil comprises a Helmholtz coil.

33. The magnetic stimulator of claim 31, wherein the at least two coils of the first coil are electrically connected to each other in series, and the at least two coils of the second coil are electrically connected to each other in series.

34. The magnetic stimulator of claim 31, wherein the at least two coils of the first coil are electrically connected to each other in parallel, and the at least two coils of the second coil are electrically connected to each other in parallel.

35. The magnetic stimulation of claim 27, wherein amplitudes of the first and second time-varying magnetic fields vary as respective sine functions.

36. The magnetic stimulator of claim 27, further comprising:
a first signal generator connected to the first coil; and
a second signal generator connected to the second coil.

37. The magnetic stimulator of claim 36, wherein:
the first signal generator produces a signal at the first frequency; and
the second signal generator produces a signal at the second frequency.

38. The magnetic stimulator of claim 27, wherein the first coil comprises:
a first conductor defining a signal path to a point on the first conductor; and
at least one second conductor defining a signal path away from the point, the at least one second conductor being electrically connected in series with the first conductor at the point and extending from the point to a location spaced from the first conductor by a predetermined distance, at least a portion of the at least one second conductor adjacent the point being non-parallel to the first conductor.

39. A magnetic stimulator for magnetically stimulating a region of a body, comprising:
a first coil producing a first time-varying magnetic field at a first frequency; and
a second coil producing a second time-varying magnetic field at a second frequency that is different than the first frequency;
wherein the first and second coils are oriented such that the first and second magnetic fields produce a beat frequency time-varying magnetic field in the region of the body, the beat frequency being between about 1 Hz and about 50 Hz and being determined by the first and second frequencies.

40. The magnetic stimulator of claim 39, wherein the beat frequency is between about 10 Hz and about 50 Hz.

41. The magnetic stimulator of claim 39, wherein the first and second frequencies are each between about 5 KHz and about 100 KHz.

42. The magnetic stimulator of claim 39, wherein the first coil comprises at least two coils, and the second coil comprises at least two coils.

43. The magnetic stimulator of claim 42, wherein the first coil comprises a Helmholtz coil, and the second coil comprises a Helmholtz coil.

44. The magnetic stimulator of claim 42, wherein the at least two coils of the first coil are electrically connected to each other in series, and the at least two coils of the second coil are electrically connected to each other in series.

45. The magnetic stimulator of claim 42, wherein the at least two coils of the first coil are electrically connected to each other in parallel, and the at least two coils of the second coil are electrically connected to each other in parallel.

46. The magnetic stimulation of claim 39, wherein amplitudes of the first and second time-varying magnetic fields vary as respective sine functions.

47. The magnetic stimulator of claim 39, further comprising:
a first signal generator connected to the first coil; and
a second signal generator connected to the second coil.

48. The magnetic stimulator of claim 47, wherein:
the first signal generator produces a signal at the first frequency; and
the second signal generator produces a signal at the second frequency.

49. The magnetic stimulator of claim 39, wherein the first coil comprises:
a first conductor defining a signal path to a point on the first conductor; and
at least one second conductor defining a signal path away from the point, the at least one second conductor being electrically connected in series with the first conductor at the point and extending from the point to a location spaced from the first conductor by a predetermined distance, at least a portion of the at least one second conductor adjacent the point being non-parallel to the first conductor.

50. A method of magnetically stimulating a region of a body, comprising:
subjecting the region to a beat frequency electric field produced by first and second time-varying magnetic fields having respective first and second frequencies, the first frequency being different than the second frequency, the region being adjacent a brain of the body.

51. The method of claim 50, further comprising:
using a first coil to generate the first time-varying magnetic field; and
using a second coil to generate the second time-varying magnetic field.

52. The method of claim 51, further comprising:
using a third coil to generate the first-time varying magnetic field; and
using a fourth coil to generate the second-time varying magnetic field.

53. The method of claim 51, wherein the first and second coils are Helmholtz coils.

54. The method of claim 50, wherein the region of the body is within the brain.

55. A method of magnetically stimulating a region of a body, comprising:
subjecting the region to a beat frequency electric field produced by first and second time-varying magnetic fields having respective first and second frequencies, the first frequency being within 50 Hz of the second frequency, the first frequency being different than the second frequency.

56. The method of claim 55, further comprising:
using a first coil to generate the first time-varying magnetic field; and
using a second coil to generate the second time-varying magnetic field

57. The method of claim 56, further comprising:
using a third coil to generate the first-time varying magnetic field; and
using a fourth coil to generate the second-time varying magnetic field.

58. The method of claim 56, wherein the first and second coils are Helmholtz coils.

59. The method of claim 55, wherein the region of the body is adjacent a brain.

60. The method of claim 59, wherein the region of the body is within the brain.

61. A method of magnetically stimulating a region of a body, comprising:
subjecting the region to a beat frequency electric field produced by first and second time-varying magnetic fields having respective first and second frequencies, the first frequency being different than the second frequency, the first and second frequencies each being between about 5 KHz and about 100 KHz.

62. The method of claim 61, further comprising:
using a first coil to generate the first time-varying magnetic field; and
using a second coil to generate the second time-varying magnetic field.

63. The method of claim 62, further comprising:
using a third coil to generate the first-time varying magnetic field; and
using a fourth coil to generate the second-time varying magnetic field.

64. The method of claim 62, wherein the first and second coils are Helmholtz coils.

65. The method of claim 62, wherein the region of the body is adjacent a brain.

66. The method of claim 65, wherein the region of the body is within the brain.

67. A method of magnetically stimulating a region of a body, comprising:
subjecting the region to a beat frequency electric field having a frequency between about 1 Hz and about 50 Hz and produced by first and second time-varying magnetic fields having respective first and second frequencies, the first frequency being different than the second frequency.

68. The method of claim 67, further comprising:
using a first coil to generate the first time-varying magnetic field; and
using a second coil to generate the second time-varying magnetic field.

69. The method of claim 68, further comprising:
using a third coil to generate the first-time varying magnetic field; and
using a fourth coil to generate the second-time varying magnetic field.

70. The method of claim 68, wherein the first and second coils are Helmholtz coils.

71. The method of claim 67, wherein the region of the body is adjacent a brain.

72. The method of claim 71, wherein the region of the body is within the brain.

73. A method of magnetically stimulating a region of a body, comprising:
generating a first time-varying magnetic field having a first frequency along a first direction oriented toward the region of the body; and
generating a second time-varying magnetic field having a second frequency along a second direction oriented toward the region of the body; wherein:
the first direction is different than the second direction;
the first frequency is different than the second frequency; and
the first frequency being within about 50 Hz of the second frequency.

74. The method of claim 73, wherein the first and second frequencies are each between about 5 KHz and about 100 KHz.

75. The method of claim 73, wherein a difference between the first and second frequencies is between about 10 Hz and about 50 Hz.

76. The method of claim 73, wherein the region of the body is adjacent a brain of the body.

77. The method of claim 73, wherein the region of the body is in a brain of the body.

78. A magnetic stimulation coil, comprising:
a first conductor defining a signal path to a point on the first conductor;
at least one second conductor defining a signal path away from the point, the at least one second conductor being electrically connected in series with the first conductor at the point and extending from the point to a location spaced from the first conductor by a predetermined distance, at least a portion of the at least one second conductor adjacent the point being non-parallel to the first conductor; and
a lead electrically connected to the second conductor.

79. The magnetic stimulation coil of claim 78, wherein the first and second conductors form an angle between about 1° and about 90° at the point.

80. The magnetic stimulation coil of claim 78, wherein the first and second conductors form an angle between about 1° and about 45° at the point.

81. The magnetic stimulation coil of claim 78, wherein the first and second conductors form an angle between about 10° and about 20° at the point.

82. The magnetic stimulation coil of claim 78, wherein respective portions of the first and second conductors proximate the point are curved.

83. The magnetic stimulation coil of claim 78, wherein the at least one second conductor comprises six conductors.

84. The magnetic stimulation coil of claim 78, wherein the second conductor comprises a wire.

85. The magnetic stimulation coil of claim 78, wherein the second conductor comprises a surface.

86. The magnetic stimulation coil of claim 78, wherein the second conductor comprises at least a portion of a cone-shaped surface.

87. The magnetic stimulation coil of claim 78, wherein the second conductor comprises at least a portion of an umbrella-shaped surface.

88. The magnetic stimulation coil of claim 78, further comprising a signal generator connected to the second conductors via the lead.

89. The magnetic stimulation coil of claim 88, wherein the signal generator operates at a frequency between about 5 KHz and about 100 KHz.

90. The magnetic stimulation coil of claim 88, wherein the signal generator operates at a frequency between about 1 Hz and about 1000 Hz.

91. The magnetic stimulation coil of claim 78, wherein the first conductor is substantially straight.

92. The magnetic stimulation coil of claim 78, wherein the first conductor comprises a helical coil.

93. The magnetic stimulation coil of claim 78, wherein:
the first conductor comprises a plurality of conductors;
the second conductor comprises a plurality of conductors; and
each of at least some of the second conductors is electrically connected in series with a different one of the first conductors.
